(19) United States
(12) Patent Application Publication
(10) Pub. No.: US 2011/0106131 A1
(43) Pub. Date: May 5, 2011

(54) Systems and Methods for Closing a Percutaneous Vascular Puncture

(75) Inventor: Jeffery C. Argentine, Petaluma, CA (US)

(73) Assignee: Medtronic Vascular, Inc., Santa Rosa, CA (US)

(21) Appl. No.: 12/608,128

(22) Filed: Oct. 29, 2009

(51) Int. Cl. A61M 29/00; A61B 17/08

(52) U.S. Cl. 606/194; 606/213

(57) ABSTRACT

A system and method for closing a percutaneous vessel puncture at the conclusion of a vascular catheterization procedure includes placement of an intravascular closure device having a tubular membrane mounted about a radially self-expandable scaffold. A tether is attached to a midpoint of the closure device and extends externally therefrom. The closure device is placed by a delivery catheter extending through the puncture site and is radially expanded in a location upstream or downstream of the puncture site. The tether extends through the vessel puncture and tension applied to the tether slides the closure device into a position covering the puncture from within the vessel.

### FIELD OF THE INVENTION

The invention relates to systems and techniques for closing a percutaneous puncture in a blood vessel at the conclusion of an intravascular catheterization procedure.

### BACKGROUND

Various cardiovascular procedures, such as angioplasty and stent placement, among others, are performed by inserting into and manipulating within a patient's vasculature, wires and catheters adapted to perform those procedures. In coronary and other such intravascular interventional procedures access to the vasculature typically is percutaneous, often through the femoral artery, involving insertion of a needle in the region of the groin to form a track through subcutaneous tissue and to puncture and create an arteriotomy in the artery. A guidewire then is advanced through the needle and into the femoral artery. The needle then is removed and a dilator carrying an introducer sheath then is advanced over the guidewire, along the needle track and into the femoral artery. The dilator enlarges the track through the tissue and widens a puncture in the vessel so that it may receive the introducer sheath, subsequent catheters and the like. With the introducer sheath having been advanced into the vessel, the dilator is removed leaving the introducer sheath in place. The guidewire and introducer sheath serve as guides to provide access into the femoral artery, through the arteriotomy, for catheters or other instrumentalities in order to perform the selected procedure within the patient's vasculature.

After the intravascular procedure has been completed, the procedural devices are removed and the arteriotomy must be closed. A number of techniques are known to facilitate closure and healing of the arteriotomy. These include application of pressure at the puncture site, often for a relatively extended length of time until hemostasis is self-sustaining, or the use of biological adhesives or plugs adapted to seal the arteriotomy, or the use of staples or clips. Some closure systems include a patch in an external position covering the arteriotomy and connected by a suture that extends through the puncture to an internal scaffold element that spans the opening. Some closure systems include an arrangement to engage the artery to temporarily draw the edges of the arteriotomy together while a final closure device, such as a staple, sutures, adhesives or other means may be used to effect the permanent closure of the arteriotomy. Some closure systems include a tubular guiding sheath that is percutaneously positioned through the enlarged needle track with a distal outlet opening of the guiding sheath disposed immediately adjacent the arteriotomy. With the sheath so positioned, a closure device can be advanced through the sheath to apply its closure element or procedure to the region of the arteriotomy to close it. In order for such a sheath-based system to be effective, it is important that the distal end of the sheath be stabilized in a fixed position relative to the vascular puncture. After the closure device has performed its function and hemostasis has been achieved, the sheath and other elements of the closure system are removed.

A challenge associated with most known vascular closure devices (VCDs) is locating the exterior surface of the vessel wall and distinguishing that surface from the surrounding subcutaneous tissue so that the closure device can be applied accurately with respect to that exterior surface. Errors in accurately determining the exterior surface of the vessel wall can result in hematoma if the VCD is deployed too far away from the vessel wall, or can result in embolization if the VCD is unintentionally deployed within the vessel lumen. It would be desirable to provide a system that can promptly and effectively achieve permanent hemostasis at a percutaneous vascular puncture without requiring the clinician to accurately locate the exterior surface of the vessel wall at an arteriotomy.

### SUMMARY OF THE INVENTION

The invention provides a closure system and methods for closing a puncture in a blood vessel, such as an arteriotomy. A delivery catheter of the system carries a tubular closure device in a radially compressed mounted configuration into the vessel lumen and deploys it to its expanded tubular configuration to lie against the inner luminal wall and cover the puncture from the interior of the vessel. The tubular closure device comprises an expandable support scaffold covered by a flexible membrane. A tether extends externally from the tubular closure device and is attached midway along the length thereof. The system also includes an external sheath that covers and maintains the closure device in its compact mounted configuration during delivery on a catheter. When the catheter is positioned to locate the sealing device at a pre-determined position in the vessel lumen upstream or downstream of the vascular puncture, the sheath is retracted and the closure device is expanded at that location. With the scaffold expanded against the vessel inner surface, the delivery catheter, sheath and guidewire are removed. Tension is applied to the tether to slide the closure device within the lumen into a position centered across the puncture. The closure device lines the luminal surface of the vessel wall and covers the puncture from within the vessel to provide hemostasis. The closure device may be made from a bioabsorbable material selected to degrade after passage of time sufficient to allow the puncture to heal naturally.

### DESCRIPTION OF THE DRAWINGS

The accompanying drawings are not intended to be in scale and in some cases are in exaggerated scale for ease of explanation and illustration.

FIG. 1 is a diagrammatic plan illustration of a portion of a blood vessel with a vascular puncture;

FIG. 2 is a diagrammatic illustration, in section, of the blood vessel as seen along the line 2-2 of FIG. 1;

FIG. 3 is a diagrammatic illustration, in section, of a blood vessel with a guidewire extending through a needle track in subcutaneous tissue, the vessel puncture and into the lumen of the vessel after an intravascular procedure has been completed but before the puncture has been closed;

FIG. 4 is a diagrammatic illustration, in section, of a blood vessel with the closure device in its fully deployed configuration, with the tubular closure device covering the puncture from the interior of the vessel;

FIG. 5 is a partially fragmented oblique illustration of a puncture closure device in accordance with the invention;

FIG. 6 is a transverse sectional view of the puncture closure device as seen along the line 6-6 of FIG. 5;

FIG. 7 is a diagrammatic illustration of the distal end of the delivery device showing the closure device;

FIG. 8 is a longitudinal sectional elevation of a portion of the delivery device as seen along the line 8-8 of FIG. 7;

FIG. 9 is a diagrammatic illustration of a vascular puncture closure system in accordance with the invention having been advanced over a guidewire and through the vascular puncture;

FIG. 10 is a diagrammatic illustration of the system in the vessel with the sheath removed to release the closure device at a selected location upstream of the puncture site; and

FIG. 11 is a diagrammatic illustration of the vascular puncture device in the vessel with the catheter removed and the tether extending from the puncture site.

### DESCRIPTION OF THE ILLUSTRATIVE EMBODIMENTS

In the description of the invention, “proximal,” will refer to a direction away from the patient, that is, toward the operator of the device, and “distal,” will refer to the opposite direction, away from the clinician and toward the patient.

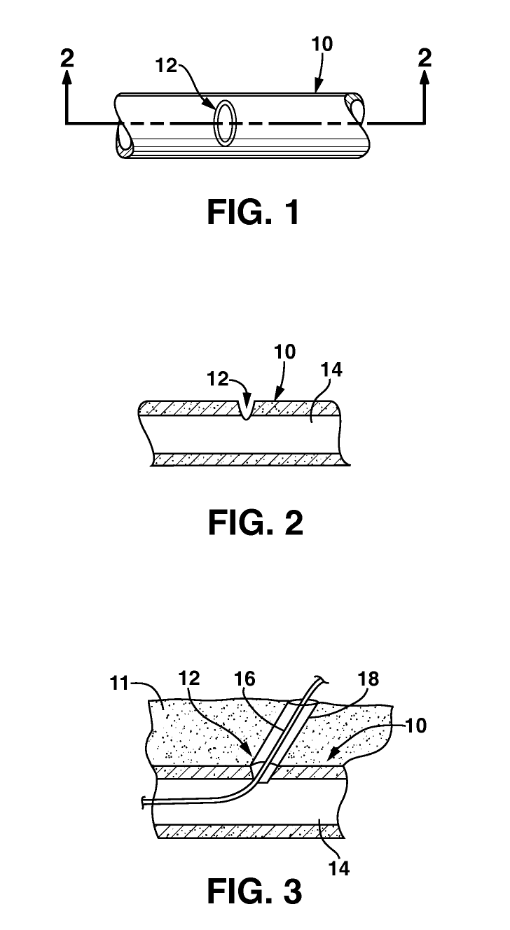

FIGS. 1-3 illustrate, diagrammatically, a segment of a blood vessel 10 (e.g., an artery) that has been punctured by a hypodermic needle (not shown) to form an arteriotomy 12 through which various wires, catheters and the like may be advanced and guided into the lumen 14 of the vessel in order to perform any of a variety of well-known intravascular procedures. As shown in FIG. 1, the typical shape of the resulting puncture in an artery is in the form of a slit that extends in a circumferential direction, resulting from the muscle structure of the artery in which the muscle fibers extend generally circumferentially. Typically, the needle puncture that initiates the arteriotomy is followed by subsequent, larger diameter instruments that progressively dilate the dimensions of arteriotomy 12 to be able to accept the larger intravascular devices. FIG. 3 illustrates the vessel 10 and a needle track 18 through tissue 11 such as skin and subcutaneous tissue by the puncture needle and with an indwelling guidewire 16 extending through the track and into vessel lumen 14, as may remain after the intravascular procedures have been completed and the last of the catheters and introducer sheath have been removed from the patient. At this point in the procedure, it is necessary to close arteriotomy 12. FIGS. 1-3 do not illustrate elements of the invention, but are intended to show an exemplary clinical environment in which the invention may be used.

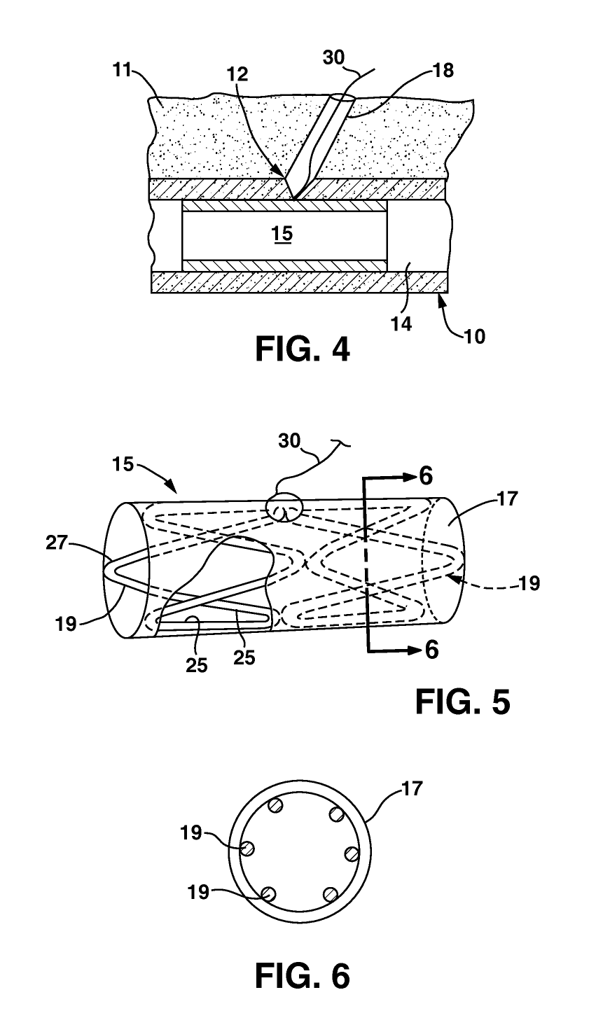

As illustrated prophetically in FIG. 4, the present invention closes arteriotomy 12 by lining the interior of blood vessel 10 with a closure device 15 (See FIGS. 5, 6) that, when deployed, has a tubular membrane 17 that lines the inner luminal surface of vessel 10 and covers puncture 12 from within the artery. The device is inserted through arteriotomy 12 by a delivery device described below. Closure device 15 includes a self-expanding tubular scaffold 19 fixedly mounted inside tubular membrane 17. The device 15 is delivered in a low-profile mounted configuration that is radially compressed and is deployed by permitting it to expand radially within the vessel lumen. Liner 17 serves to cover arteriotomy 12 from within blood vessel 14 to enable the arteriotomy to heal naturally while maintaining hemostasis. Closure device 15 may be formed from bioabsorbable materials selected to be absorbed by the body after a sufficient time has passed to permit healing of the vessel puncture site. Suitable bioabsorbable materials may include poly-alpha-hydroxy acids such as polyglycolic acid (PGA), polylactic acid, copolymers of lactic and glycolic acids, and such polymers copolymerized with ε-caprolactone or trimethylene carbonate. Stiffer bioabsorbable materials may be utilized as fine fibers in braided tubes or non-oriented tubular fibrous mats wherein the porosity of liner 17 is small to begin with, and which will quickly be sealed by clotting. More flexible materials, e.g. glycolide copolymers, may be utilized in solid tubular form.

The tubular scaffold 19 may take any of a number of known configurations, such as a radially expandable stent-like device. Tubular membrane 17 may be attached, as by suturing or adhesive or thermal bonding to scaffold 19 directly at a plurality of locations that will allow both scaffold 19 and membrane 17 to expand radially from their low profile configuration on the delivery catheter to an expanded, deployed condition in slidable engagement with the inner luminal surface of the vessel (FIG. 4).

FIGS. 5 and 6 illustrate a type of scaffold 19 similar to one or more modules of a zigzag type of stent known to those in the art. In the illustrated example, scaffold 19 is formed from a two conjoined wire-like structures, each defined by alternating struts 25 joined end-to-end or formed into bends 27. In this embodiment, the distal end of tubular membrane 17 may be attached at a number of individual points, such as at the bends 27 of the zigzag configuration. Other configurations known from self-expanding stents may be adapted to scaffold 19, either wireform types or those having a pattern cut from a solid-walled tube. Tubular membrane 17 may be formed from a thin biodegradable film. The scaffold 19, however, if formed from a metal, e.g. nitinol, may remain implanted in the artery after the puncture 12 has healed and the tubular membrane 17 has been absorbed. The scaffold 19 also may be formed from a biodegradable polymer capable of expansion to a radially expanded size that will retain itself within the vessel during deployment and subsequent degradation. Membrane 17 may be formed from biocompatible materials that are suitable for medical implants, but are not bioabsorbable, e.g. expanded polytetrafluoroethylene (EPTFE).

Puncture closure device 15 and the associated delivery system provide for closing the puncture 12 through which it is delivered. Compacted closure device 15 is to be deployed upstream or downstream of the arteriotomy 12 and tends to self-expand to a pre-formed or relaxed diameter that is larger than the diameter of the vessel lumen 14. Known stents or stent-grafts are expected to engage the vessel wall with sufficient friction to remain in the location where they are implanted. Unlike those devices, closure device 15 is expected to have low friction between membrane 17 and the inner surface of vessel 10 to permit controlled axial sliding of the device within the blood vessel after the delivery device has been withdrawn. Thus, closure device 15 is expected to be released against the vessel wall at some distance from arteriotomy 12 through which it is delivered; then the device is slid into a position covering arteriotomy 12 from within the vessel. Some features that may be employed to provide low friction between membrane 17 and the inner surface of vessel 10 include using a low-friction material for membrane 17, e.g. EPTFE, having a short length to provide a small contact area, and having only a light interference fit, i.e. the relaxed or expanded diameter of closure device 15 being only slightly greater than the diameter of the vessel lumen 14 in which the device is implanted.

Tether 30 is used to slide closure device 15 into closure position covering arteriotomy 12 from within the vessel. Tether 30 is a flexible filament such as a suture attached to scaffold 19 and extending outwardly through membrane 17. Tether 30 may be attached to scaffold 19 by a tied knot or any other suitable means. Optionally, tether 30 may be looped through the attachment point on scaffold 19 such that two free ends extend from the patient (not shown). In order to slide closure device into an approximately centered position across puncture 12, tether 30 is attached to scaffold 19 at a location that is longitudinally centered or is spaced at least some distance from either end of closure device 15.

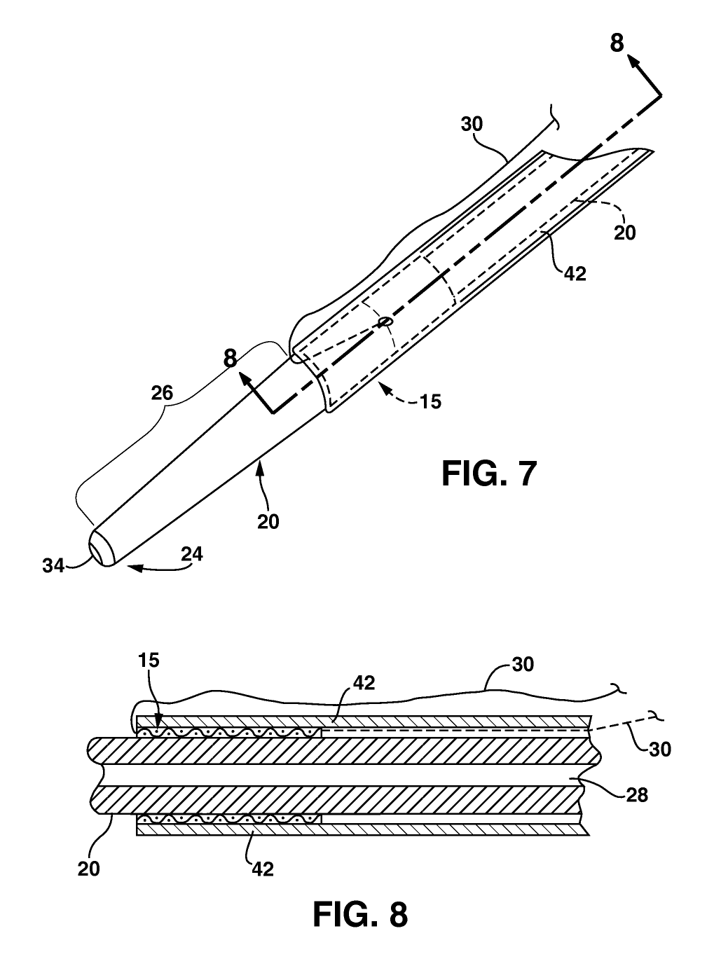

FIGS. 7 and 8 depict, somewhat diagrammatically, an illustrative embodiment of a delivery device for use in the practice of the invention. The device includes a catheter 20 that may be formed as an elongate flexible shaft, as by extrusion, from any of a variety of polymers commonly used in the construction of catheter shafts, such as PEBAX® polyethylene block amide co-polymer from ARKEMA, Philadelphia, Pa. Catheter 20 has a proximal end (not shown) and a distal end 24, the distalmost portion of the shaft having a taper 26 to facilitate passage along subcutaneous needle track 18 and through vascular puncture 12. In the illustrative embodiment, catheter 20 has a guidewire lumen 28 extending from the proximal end of the catheter and terminating in a distal opening 34 at distal end 24 of catheter 20. The proximal end of catheter 20 may include a fitting (not shown) that may be molded directly onto the shaft, as is common practice in the art of medical catheters.

The delivery device also includes an external tubular sheath 42 that is slidably disposed on catheter 20. The distal portion of the sheath 42 overlies and contains closure device 15, maintaining it in a low profile during delivery. Tether 30 extends from an open distal end of sheath 42 and may trail freely alongside the delivery device. Optionally, tether 30 may extend proximally between catheter 20 and sheath 42, as shown in the alternative position in FIG. 8, to exit at the proximal end of the delivery device. Sheath 42 has a length that is less than that of catheter 20 and has a proximal end that allows the sheath to be withdrawn proximally over and/or torn away from catheter 20 to expose closure device 15. When sheath 42 is in its distal position on catheter 20 with its distal end overlying closure device 15, the delivery device should be advanced distally in the vessel to assure that the entire closure device 15 is within the vessel and distally beyond puncture 12. The intended distal location may be either upstream or downstream of puncture 12.

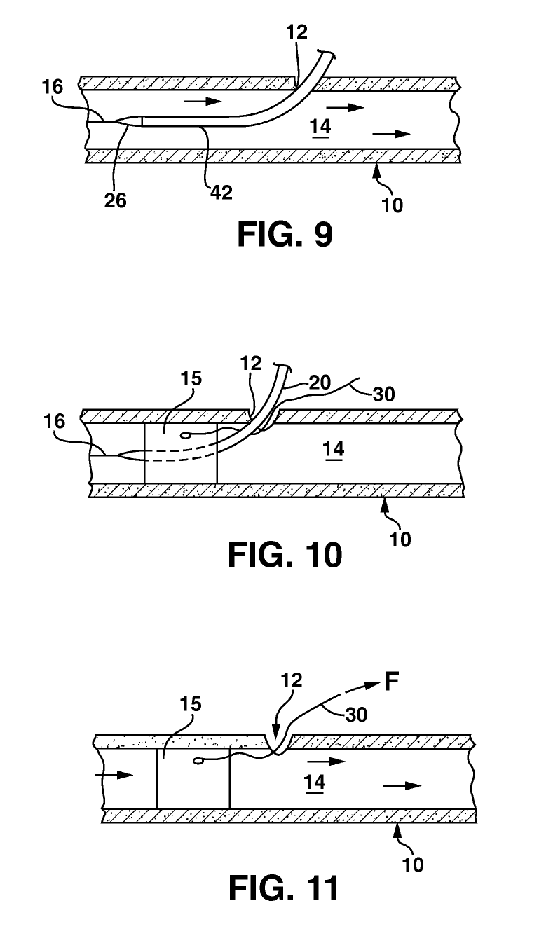

The closure system is used in a manner illustrated prophetically in FIGS. 4, and 9-11. After the intravascular procedure has been completed and the associated interventional or diagnostic catheters have been removed, leaving only the indwelling guidewire 16 in place (FIG. 3), the closure system containing the closure device 15 is backloaded onto the proximal end of the externally accessible guidewire 16. The closure system, guided by the guidewire 16, is advanced through the needle track and vessel puncture 12 to position the closure device 15 distally of the vascular puncture 12. Sheath 42 then is retracted proximally to expose closure device 15 (FIG. 10). Closure device 15 is self-expanded radially into engagement with the inner luminal wall of the vessel 10. The delivery device and the guidewire are removed through the puncture 12, leaving the radially expanded closure device 15 temporarily located within the blood vessel lumen with tether 30 extending through puncture 12 (FIG. 11) and out of the patient. Tension force F is applied to tether 30 to slide closure device 15 into a sealing position covering puncture 12 from the inside of vessel 10 (FIG. 4). Tether 30 maybe affixed to tissue 11, e.g. by using an adhesive bandage or by placing a stitch into skin or subcutaneous tissue. If closure device 15 is considered by the clinician to be sufficiently secured in the sealing position by the amount of friction between membrane 17 and the inner surface of vessel 10, then tether 30 may be substantially removed, e.g. by a releasing a slipknot at scaffold 19, by releasing one free end of the tether and pulling it out by the other free end, or by cutting tether 30 below skin level in needle track 18 to leave only a small tether portion in the patient. Scaffold 19, membrane 17 and tether 30 may be formed from bioabsorbable materials that, over time, allow the puncture wound to heal naturally.

The dimensions of a device in accordance with the invention will, of course, depend on the size of the vessel in which it is to be used, and the size of the puncture 12 being closed. The closure system can be smaller than the puncture, and to avoid further enlargement of the puncture, it is preferable for the closure system to be no larger in diameter than the largest device that was used during the catheterization procedure. For example, in the case of a puncture in the femoral artery for implantation of a stent-graft for treatment of an aortic aneurysm, sheath 42 of the delivery device may have an outer diameter in a range from about 10 French (0.131 inch) to 12 French (0.157 inch). Other sheath diameters, both smaller and larger than this example, may be suitable for systems used in closing vascular punctures after different catheterization procedures. In one example, the scaffold 19 may be of the order of about 0.070 inch outer diameter when in its low profile configuration. It should be self-expandable to a relaxed diameter of up to about 0.350 inch to be usable in a vessel having an inner diameter of up to about 0.314 inch (8 millimeters). The length of the closure device may be approximately two to two and a half times the relaxed diameter.

It should be understood that the foregoing description of the invention is intended merely to be illustrative and that other embodiments and equivalents may be employed within the scope of the invention.

What is claimed is:

1. A system for closing a percutaneous puncture into a lumen defined by the inner surface of a blood vessel wall, the system including:
a tubular closure device having open ends and a relaxed configuration having a diameter larger than the lumen of the blood vessel, the closure device comprising:
a tubular membrane;
a self-expanding tubular scaffold fixed within the tubular membrane; and
a tether attached to the scaffold at a location spaced from either end of the closure device and extending externally through the membrane;
a delivery catheter comprising an elongate flexible shaft having a proximal end and a tapered portion at a distal end, the closure device being mounted in a radially compressed configuration about the delivery catheter; and
a tubular sheath having an initial position disposed about the shaft and the closure device, the sheath being retractable from its initial position proximally to release the closure device into the relaxed configuration wherein the scaffold and the membrane are radially expanded;
wherein, when the closure device is in the relaxed configuration within the vessel lumen, a friction force between the membrane of the closure device and the inner surface of the blood vessel wall can be overcome by tension applied to the tether to slide the closure device axially in the blood vessel.

2. The system of claim 1 further comprising a guidewire lumen extending from the shaft proximal end to an axial opening in the shaft distal end.

3. The system of claim 1 wherein at least a portion of the closure device is biodegradable.

4. The system of claim 1 wherein the tubular scaffold comprises at least one expandable ring and wherein the membrane is attached to the ring at a plurality of locations whereby both the scaffold and the membrane can expand together radially to transform the closure device from the radially compressed configuration to the relaxed expanded configuration.

5. The system of claim 4 wherein the at least one expandable ring comprises a zig-zag wireform.

6. The system of claim 1 wherein the tether is attached to the scaffold at a midpoint along the length of the closure device.

7. The system of claim 1 wherein, when the sheath is in the initial position, the tether extends out of an open distal end of the sheath.

8. A method for closing a puncture of a blood vessel following a percutaneous catheterization thereof, the method comprising:
receiving a closure device having a tubular membrane attached around a tubular scaffold and a tether being attached at a location spaced from either end of the closure device and extending externally therefrom, the closure device being radially expandable from a low profile configuration to a relaxed diameter;
inserting the closure device through the puncture and advancing it in the vessel lumen to locate the closure device at a distance from the puncture;
releasing the closure device to radially self-expand into contact with a wall of the blood vessel such that the tether extends from the closure device through the vessel lumen and externally through the puncture; and
pulling on the tether to slide the closure device axially into a position covering the puncture from within the vessel.

9. The method of claim 8 wherein the step of receiving the closure device further comprises receiving a delivery assembly wherein the closure device is mounted in the low profile configuration about an elongate shaft having a guidewire lumen, and wherein an external sheath is disposed about the shaft to enclose the closure device.

10. The method of claim 9 wherein the step of inserting the closure device further comprises:
advancing the delivery assembly over a percutaneously placed guidewire that extends into the vessel through the puncture; and
after locating the closure device at a selected position away from the puncture, withdrawing the sheath proximally to uncover and release the closure device.

11. The method of claim 8 wherein the step of receiving a delivery assembly further comprises receiving a delivery assembly wherein the closure device is disposed within the external sheath such that the tether extends out of an open distal end of the sheath.

12. The method of claim 8 wherein at least a portion of the closure device is biodegradable.
